(19) United States
(12) Patent Application Publication
(10) Pub. No.: US 2009/0156876 A1
(43) Pub. Date: Jun. 18, 2009

(54) Apparatus and Process for Cracking Hydrocarbonaceous Feed Treated to Adsorb Paraffin-Insoluble Compounds

(76) Inventors: John D. Y. Ou, Houston, TX (US); Robert D. Strack, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/958,433

(22) Filed: Dec. 18, 2007

(51) Int. Cl. C07C 7/12; B01J 19/00

(52) U.S. Cl. 585/820; 422/211

(57) ABSTRACT

An apparatus and process are provided for thermally cracking hydrocarbon feeds containing paraffin insoluble compounds by selectively adsorbing paraffin-insoluble compounds with solid particulate adsorbents, prior to cracking. An aromatics-containing stream such as one derived from cracked product is used to desorb the paraffin insoluble compounds from the adsorber stage.

### FIELD OF THE INVENTION

The present invention relates to the cracking of hydrocarbons, especially with feeds containing relatively non-volatile hydrocarbons, e.g., paraffin-insoluble compounds. More particularly, the present invention relates to a cracking apparatus and process which selectively adsorbs paraffin-insoluble compounds from cracker feedstock in an adsorber stage prior to cracking. The invention further relates to an apparatus and process which utilizes an aromatic-containing process stream derived from cracked product to desorb the paraffin insoluble compounds from the adsorber stage.

### BACKGROUND OF THE INVENTION

Steam cracking, also referred to as pyrolysis, has long been used to crack various hydrocarbon feedstocks into olefins, preferably light olefins such as ethylene, propylene, and butenes. Conventional steam cracking utilizes a pyrolysis furnace that has two main sections: a convection section and a radiant section. The hydrocarbon feedstock typically enters the convection section of the furnace as a liquid (except for light feedstocks which enter as a vapor) wherein it is typically heated and vaporized by indirect contact with hot flue gas from the radiant section and by direct contact with steam. The vaporized feedstock and steam mixture is then introduced into the radiant section where the cracking takes place. The resulting products including olefins leave the pyrolysis furnace for further downstream processing.

Pyrolysis involves heating the feedstock sufficiently to cause thermal decomposition of the larger molecules. The pyrolysis process, however, produces molecules that tend to combine to form high molecular weight materials known as tar. Tar is a high-boiling point, viscous, reactive material that can foul equipment under certain conditions. In general, feedstocks containing higher boiling materials tend to produce greater quantities of tar.

Conventional steam cracking systems have been effective for cracking high-quality feedstock which contains a large fraction of light volatile hydrocarbons, such as gas oil and naphtha. However, steam cracking economics sometimes favor cracking lower cost heavy feedstocks such as, by way of non-limiting examples, crude oil and atmospheric residue. Crude oil and atmospheric residue often contain high molecular weight, non-volatile components with boiling points in excess of about 590° C. (1100° F.) otherwise known as resids. The non-volatile components of these feedstocks lay down as coke in the convection section of conventional pyrolysis furnaces. Only very low levels of non-volatile components can be tolerated in the convection section downstream of the point where the lighter components have fully vaporized.

Cracking heavier feeds, such as kerosenes and gas oils, produces large amounts of tar, which lead to rapid coking in the radiant section of the furnace as well as fouling in the transfer line exchangers preferred in lighter liquid cracking service.

Additionally, during transport some naphthas are contaminated with heavy crude oil containing non-volatile components. Conventional pyrolysis furnaces do not have the flexibility to process residues, crudes, or many residue or crude contaminated gas oils or naphthas which are contaminated with non-volatile components.

Non-volatile components in pyrolysis feedstocks typically contain paraffin-insoluble compounds, such as pentane-insoluble (PI) compounds or heptane-insoluble (HI) compounds, which are molecules of high molecular weight with multi-ring structures, e.g., asphaltenes. These materials can be present in various hydrocarbon streams as naturally-occurring components, contaminants, e.g., those introduced during transport, and/or by-products formed during feed processing, e.g., during cracking. The paraffin-insoluble compounds are particularly undesirable given their proclivity to form tar or coke during processing. Moreover, their presence reduces the economic value of a hydrocarbon stream by rendering it less compatible for mixing with highly paraffinic streams, inducing precipitation of the paraffin-insoluble components from the resulting mixture.

Various methods are known in the art to treat various feedstocks to reduce the content of paraffin-insoluble compounds.

U.S. Pat. No. 4,634,516 to Haskell et al., incorporated herein by reference in its entirety, discloses treating gas oil or kerosene feeds to a steam cracker, removing aromatic coke precursors by contacting the feed with 4 to 50 mesh size activated carbon particulates to form a slurry from which the particulates are removed prior to steam cracking. The particulates absorb polynuclear aromatics and can be regenerated in the presence of steam and carbon dioxide for reuse.

U.S. Pat. No. 4,804,457 to Ngan, incorporated herein by reference in its entirety, discloses multi-stage reforming of hydrocarbon with intermediate adsorption zones for adsorbing polynuclear aromatics formed in each stage. The adsorbent zone treats gas oil or kerosene feeds to a steam cracker, removing aromatic coke precursors with adsorption sieves selected from molecular sieve, silica gel, silica, alumina, activated alumina, activated carbon, silica-alumina and various clays.

U.S. Pat. No. 6,245,223 to Gorbaty et al., incorporated herein by reference in its entirety, discloses removing metals and coke precursors from a catalytic cracker feed such as vacuum residua, vacuum gas oils, solvent deasphalting fractions (DAO using deasphalting solvent such as propane, butane, or pentane) and whole crudes. The cracker feed is contacted with a hydrocarbon insoluble adsorbent in a fixed, ebullating, or slurry bed. Effluent from the adsorbent bed reduced in coke precursors is passed to a catalytic cracker. The adsorbent can be regenerated using solvents such as toluene and toluene-methanol to wash metal containing and coke precursor molecules off the adsorbent. The adsorbent can be provided in two separate vessels to permit swing operation with adsorption in one and regeneration in the other.

U.S. Pat. No. 5,583,277 to Kuehl, incorporated herein by reference in its entirety, discloses removal of large molecules such as polynuclear aromatics from waste or process streams, e.g., reformate, using an M41S material, e.g., MCM-41 as an adsorbent.

U.S. Pat. No. 4,775,460 to Reno, incorporated herein by reference in its entirety, discloses pretreating hydrocracker feed by contacting with a metal-free alumina to produce polycyclic compounds or their precursors, followed by contacting the feed with a bed of adsorbent such as charcoal, silica gels, and large pore aluminas. After hydrocracking, the treated feed will have a lower concentration of polycyclic aromatic hydrocarbons than prior art processes.

U.S. Pat. No. 4,447,315 to Lamb et al., incorporated herein by reference in its entirety, discloses hydrocracking a feedstock to provide a hydrocrackate containing polynuclear aromatic compounds, recycling at least a portion of unconverted hydrocarbon oil or recycle stream containing polynuclear aromatic compounds to contact an adsorbent which selectively retains polynuclear aromatic compounds to remove essentially all of the PNAs from the recycle hydrocarbon stream. The PNA-depleted stream is then directed to the hydrocracker. The adsorbent, e.g., molecular sieves, silica gel, activated carbon, activated alumina, silica-alumina gel, and clays, may be installed in one or more vessels, e.g., as two fixed beds in swing arrangement.

It would be desirable to provide an apparatus and process for treating feeds for cracking that contain paraffin-insoluble compounds by contact with an adsorbent selective for paraffin-insoluble compounds. Moreover, it would be particularly desirable to provide such an apparatus and process that can utilize readily available aromatics-containing streams to desorb used adsorbent, especially for providing operations that are essentially self-contained insofar as they provide for regeneration of the adsorbent utilizing a desorber stream derived from the cracking process itself.

### SUMMARY OF THE INVENTION

It has now been found that aromatics-containing streams, such as those derived from cracking, e.g., pyrolysis cracking, are capable of providing a desorber stream suitable for desorbing paraffin-insoluble compounds from adsorbent particles used to treat cracking feeds that contain paraffin-insoluble compounds.

In one aspect, the present invention relates to an apparatus for cracking hydrocarbonaceous feed containing paraffin-insoluble compounds, which comprises: A) a feed treating zone, which comprises an adsorber vessel for treating the hydrocarbonaceous feed comprising a) a hydrocarbonaceous feed inlet, b) adsorbent particles capable of selectively sorbing paraffin-insoluble compounds, and c) an outlet for removing paraffin-insoluble compounds-depleted effluent; B) a cracking zone comprising a) an inlet for receiving paraffin-insoluble compounds-depleted effluent from the feed treating zone, b) an outlet for removing cracked effluent containing aromatics; C) a separator for removing an aromatics-containing stream from the cracked effluent comprising a) an inlet for receiving the cracked effluent, b) an outlet for removing the aromatics-containing stream, and c) an outlet for removing remaining cracked effluent; and D) an inlet to the feed treating zone for receiving aromatics-containing stream as desorbent from the outlet for removing the aromatics-containing stream of step C).

In an embodiment of this aspect of the invention, the present invention relates to an apparatus wherein the separator C) comprises a primary fractionator.

In another embodiment of this aspect of the invention, the apparatus further comprises E) a tar knockout drum between B) and C), comprising a) an inlet for receiving cracked effluent containing aromatics, b) a bottom outlet for removing tar and c) an upper outlet for directing tar-depleted effluent to the primary fractionator. Typically, the primary fractionator comprises a) an inlet for receiving the tar-depleted effluent, b) a bottoms outlet for removing fractionator bottoms, and c) at least one outlet for removing lower boiling products. The primary fractionator can further comprise d) an overhead outlet, e) an upper outlet for removing steam-cracked naphtha and f) a lower outlet for removing steam-cracked gas oil.

In still another embodiment of this aspect of the invention, at least one of the upper outlet for removing steam-cracked naphtha and lower outlet for removing steam-cracked gas oil communicates with D) to provide an aromatics-containing desorber stream.

In still yet another embodiment of this aspect of the invention, the feed treating zone comprises at least two adsorption vessels arranged to allow selectively sorbing paraffin-insoluble compounds by adsorbent particles in one vessel while regenerating adsorbent particles in another vessel to provide an aromatics-containing stream containing desorbed paraffin-insoluble compounds, each vessel having an outlet for removing the aromatics-containing stream containing desorbed paraffin-insoluble compounds.

In yet still another embodiment of this aspect of the invention, the apparatus comprises an outlet for each adsorption vessel, for removing the aromatics-containing stream containing desorbed paraffin-insoluble compounds, which outlet communicates with the tar knockout drum inlet.

In another embodiment of this aspect of the invention, the bottoms outlet for removing primary fractionator bottoms communicates with the tar knockout drum inlet.

In still another embodiment of this aspect of the invention, the paraffin-insoluble compounds are selected from the group consisting of pentane-insoluble compounds and heptane-insoluble compounds.

In yet another embodiment of this aspect of the invention, the adsorbent particles are selected from the group consisting of activated carbon and inorganic oxides, say, e.g., the adsorbent particles which contain components selected from the group consisting of crystalline inorganic oxide molecular sieve, e.g., MCM-41, activated alumina, silica, aluminosilicate, clay, and fuller's earth.

In still yet another embodiment of this aspect of the invention, the hydrocarbonaceous feed containing paraffin insoluble compounds is selected from the group consisting of condensate and gas oil.

In yet still another embodiment of this aspect of the invention, the apparatus comprises a line for recycling the paraffin-insoluble compounds-depleted effluent to the hydrocarbonaceous feed inlet of the adsorber vessel.

In another aspect, the present invention relates to a process for cracking hydrocarbonaceous feed containing paraffin insoluble compounds that comprises: a) contacting the feed in an adsorption zone with adsorbent particles capable of selectively adsorbing paraffin insoluble compounds from the feed, under conditions sufficient for the adsorbent particles to sorb the paraffin-insoluble compounds to provide a treated feed of reduced paraffin-insoluble compounds content and adsorbent particles at least partially sorbed with paraffin-insoluble compounds; b) cracking the treated feed in a cracking zone at conditions sufficient to provide a cracked product containing aromatics; c) separating an aromatics-containing stream from the cracked product; and d) directing the aromatics-containing stream from the cracked product to the adsorption zone and contacting with the adsorbent particles under desorption conditions sufficient to at least partially desorb the paraffin-insoluble compounds therefrom, thereby providing regenerated adsorbent particles and an aromatics-containing stream containing desorbed paraffin-insoluble compounds.

In an embodiment of this aspect of the invention, the process further comprises: e) separating the regenerated adsorbent particles from the aromatics-containing stream containing desorbed paraffin-insoluble compounds.

In another embodiment of this aspect of the invention, the process further comprises: f) separating the paraffin-insoluble compounds from the aromatics-containing stream containing desorbed paraffin-insoluble compounds.

In still another embodiment of this aspect of the invention, the process further comprises: g) recycling the aromatics-containing stream of step f) to step d).

In another embodiment of this aspect of the invention, the process further comprises: h) recycling at least a portion of the treated feed to the adsorption zone.

In yet another embodiment of this aspect of the invention, the adsorbent particles are selected from the group consisting of activated carbon and inorganic oxides, and the paraffin-insoluble compounds are selected from the group consisting of pentane-insoluble compounds and hexane-insoluble compounds.

In still yet another embodiment of this aspect of the invention, the adsorbent particles are selected from the group consisting of MCM-41, activated alumina, silica, aluminosilicate, clay, and fuller's earth.

In yet still another embodiment of this aspect of the invention, the adsorption zone comprises at least two adsorption vessels which provide for simultaneous operation of steps a) and d) in separate adsorption vessels.

In another embodiment of this aspect of the invention, the aromatics-containing stream from the lower boiling cracked product is selected from the group consisting of gasoline, toluene, and xylene.

In still another embodiment of this aspect of the invention, the aromatics-containing stream from the cracked product is selected from the group consisting of steam-cracked naphtha, steam-cracked gas oil, and steam-cracked quench oil.

In yet another embodiment of this aspect of the invention, the separation in step f) is carried out in a tar knockout drum.

In still yet another embodiment of this aspect of the invention, the separation in step f) is carried out in a primary fractionator.

In yet still another embodiment of this aspect of the invention, the hydrocarbonaceous feed containing paraffin-insoluble compounds is selected from the group consisting of condensate and gas oil.

In another aspect, the present invention relates to an apparatus for steam cracking hydrocarbonaceous feed containing paraffin-insoluble compounds, which comprises: A) a feed treating zone, which comprises an adsorber vessel for treating the hydrocarbonaceous feed comprising a) a hydrocarbonaceous feed inlet, b) adsorbent particles capable of selectively sorbing paraffin-insoluble compounds, and c) an outlet for removing paraffin-insoluble compounds-depleted effluent; B) a steam cracking zone comprising a) an inlet for receiving paraffin-insoluble compounds-depleted effluent from the feed treating zone, b) an outlet for removing cracked effluent containing aromatics, and c) a steam inlet; and C) a separator for removing an aromatics-containing stream from the cracked effluent comprising a) an inlet for receiving the cracked effluent, b) an outlet for removing the aromatics-containing stream, and c) an outlet for removing remaining cracked effluent.

In one embodiment of this aspect, the adsorber vessel further comprises: at least one of d) an inlet to the feed treating zone for receiving aromatics-containing stream as desorbent, and e) a line for recycling paraffin-insolubles compounds-depleted effluent from outlet c) to hydrocarbonaceous feed inlet a).

In another aspect, the present invention relates to a process for steam cracking hydrocarbonaceous feed containing paraffin-insoluble compounds that comprises: a) contacting the feed in an adsorption zone with adsorbent particles capable of selectively adsorbing paraffin insoluble compounds from the feed under conditions sufficient for the adsorbent particles to sorb the paraffin-insoluble compounds to provide a treated feed of reduced paraffin-insoluble compounds content and adsorbent particles at least partially sorbed with paraffin-insoluble compounds; b) steam cracking the treated feed in a cracking zone at conditions sufficient to provide a cracked product containing aromatics; and c) separating an aromatics-containing stream from the cracked product.

In one embodiment of this aspect, the process further comprises: d) directing an aromatics-containing stream to the adsorption zone and contacting with the adsorbent particles under desorption conditions sufficient to at least partially desorb the paraffin-insoluble compounds therefrom, thereby providing regenerated adsorbent particles and an aromatics-containing stream containing desorbed paraffin-insoluble compounds.

In another embodiment of this aspect, the process further comprises e) recycling at least a portion of the treated feed of reduced paraffin-insoluble compounds content to the adsorption zone.

### BRIEF DESCRIPTION OF THE DRAWINGS

FIG. 1 depicts a process arrangement and apparatus for paraffin-insolubles removal in a steam cracking plant environment, in accordance with the present invention.

### DETAILED DESCRIPTION OF THE INVENTION

Unless otherwise stated, all percentages, parts, ratios, etc. are by weight. Ordinarily, a reference to a compound or component includes the compound or component by itself, as well as in combination with other compounds or components, such as mixtures of compounds.

Further, when an amount, concentration, or other value or parameter is given as a list of upper preferable values and lower preferable values, this is to be understood as specifically disclosing all ranges formed from any pair of an upper preferred value and a lower preferred value, regardless of whether ranges are separately disclosed.

Suitable hydrocarbonaceous feeds for use in the present invention include naphtha boiling range materials, as well as those boiling with a final boiling point in a temperature range from above about 180° C. (356° F.), such as feeds heavier than naphtha. Such feeds include those boiling in the range from about 93° C. to about 649° C. (from about 200° F. to about 1200° F.), say, from about 204° C. to about 510° C. (from about 400° F. to about 950° F.). Typical heavier than naphtha feeds can include heavy condensates, gas oils, kerosene, hydrocrackates, crude oils, and/or crude oil fractions. Preferably, the hydrocarbonaceous feed is selected from low sulfur waxy residue, crude, vacuum resid, hydrotreated atmospheric resid, hydrotreated vacuum resid, and hydrotreated crude.

The hydrocarbonaceous feeds can comprise a large portion, such as from about 5% to about 50%, of non-volatile components, i.e., resid. Such feeds could comprise, by way of non-limiting examples, one or more of steam cracked gas oils and residues, gas oils, heating oil, jet fuel, diesel, kerosene, gasoline, catalytically cracked naphtha, hydrocrackate, reformats, raffinate reformate, distillate, virgin naphtha, atmospheric pipestill bottoms, vacuum pipestill streams including bottoms, wide boiling range naphtha to gas oil condensates, heavy non-virgin hydrocarbon streams from refineries, vacuum gas oils, heavy gas oil, naphtha contaminated with crude, atmospheric residue, heavy residue, C₄'s/residue admixture, naphtha/residue admixture, hydrocarbon gases/residue admixture, hydrogen/residue admixtures, gas oil/residue admixture, and crude oil. Suitable whole crude oils include those containing high levels of nickel and vanadium such as found in Venezuela tars, for example. Solvent deasphalted (or deashpaltened) (SDA) fractions with and without resins, are especially suited for use as feedstocks in the present invention. The foregoing hydrocarbonaceous feeds can have a nominal end boiling point of at least about 315° C. (600° F.), generally greater than about 510° C. (950° F.), typically greater than about 590° C. (1100° F.), for example, greater than about 760° C. (1400° F.). The economically preferred feedstocks are generally low sulfur waxy residues, atmospheric residues, naphthas contaminated with crude, various residue admixtures and crude oil.

Investigation of the mechanism of the carbon formation in lube oils led to the discovery that, on air oxidation of a baseoil, paraffin-insoluble compounds are the first molecular species formed in the baseoils upon oxidation, and that these compounds are transformed gradually into carbonaceous material containing high infusable coke (quinoline insolubles). During oxidative-polymerization of a baseoil at high temperatures, e.g., 240° C.-360° C. (464° F.-680° F.), portions of the baseoils will react with oxygen and other oxidizing agents, e.g., sulfur, leading to polymerization and introduction of various oxygen functional groups such as phenolic, hydroxyl, carboxyl, ketones, aldehydes, ethers, etc. Other polar atoms such as sulfur and nitrogen are also present. These high molecular weight, highly oxidized molecules become insoluble in aliphatic solvents and can be determined quantitatively as the insolubles in paraffinic solvents. This insoluble portion can be referenced herein as asphaltenes.

Asphaltenes generally are composed of carbon, hydrogen, oxygen, sulfur with a C:H atomic ratio of 1.0-1.5 and average molecular weight of about 250-1000. They are brownish solids with melting points of 100° C.-400° C. (212° F.-752° F.), with extremely high tendency to coke formation at 200° C.-300° C. (392° F.-572° F.) in a non-oxygen nitrogen atmosphere with a coke yield of 35%-55% over 2 hrs. The asphaltenes have a decomposition temperature of about 400° C. (752° F.) as determined by thermogravimetric analysis in nitrogen (heating rate 10° C./minute).

Among the wide range of paraffin insolubles which are formed upon heating and oxidation, the pentane-insolubles and heptane-insolubles, hereinafter designated as C₅-asphaltenes and C₇-asphaltenes, are of particular interest. Asphaltenes may be specified with reference to the particular paraffins in which they are insoluble, e.g., n-heptane, n-hexane, n-pentane, isopentane, petroleum ether, etc. For present purposes, asphaltene content of a sample can be determined by well-known analytic techniques, e.g., ASTM D6560 (Standard Test for Determination of Asphaltenes (Heptane Insolubles) in Crude Petroleum and Petroleum Products), ASTM D3270 (Standard Test Method for n-Heptane Insolubles), ASTM D4055-02 (Standard Test Method for Pentane Insolubles by Membrane Filtration), and ASTM D-893 (Standard Test Method for Insolubles in Used Lubricating Oils).

The hydrocarbonaceous feed intended for cracking is subjected to an adsorption process by contacting with a hydrocarbon insoluble adsorbent material, which has a high capacity for adsorbing metals and coke precursors, and which can be easily regenerated by washing with aromatics-containing streams. In the present invention, hydrocarbonaceous feed prior to adsorption can contain at least about 0.001 wt % C₅-asphaltenes and/or C₇-asphaltenes, say, from about 0.001 wt % to about 0.2 wt %, typically from about 0.01 wt % to about 0.2 wt %.

The hydrocarbonaceous feed to be cracked is contacted in an adsorption zone with adsorbent particles capable of selectively adsorbing paraffin-insoluble compounds from the feed. The adsorption zone typically comprises one or more vessels of sufficient volume to allow adequate contact of the feed to be treated with the adsorbent particles. The vessel may contain a suitable mechanical mixing means to promote contact of the hydrocarbonaceous feed and adsorbent particles.

A suitable adsorber vessel comprises an inlet for the hydrocarbonaceous feed, and an outlet for removing paraffin-insoluble compounds-depleted effluent, which are utilized during adsorption. An aromatics-containing desorber stream for regenerating adsorbent particles can be provided by a separate inlet or the same inlet used for the hydrocarbonaceous feed. Similarly, a separate outlet can be provided for removing spent stream aromatics-containing streams that also contain paraffin-insoluble compounds after contact with the adsorbent particles; or the same outlet used for removing paraffin-insoluble compounds-depleted effluent can be used for this purpose. The adsorber vessel can also comprise a separate inlet/outlet for adding/removing adsorbent particles to the vessel. Alternately, the adsorbent particles can be added through other inlet(s) and outlet(s) of the vessel. The adsorbent may be installed in the adsorption zone in any suitable manner. The vessel can be configured as a fixed bed, ebullating bed or slurry bed. All of these beds are well-known in the art. The adsorbent may be installed in one or more vessels and in either series or parallel flow. The flow of hydrocarbons through the adsorption zone is preferably performed in a parallel manner so that when one of the adsorbent beds or chambers is spent by the accumulation of paraffin-insoluble compounds thereon, the spent zone may be bypassed while continuing uninterrupted operation through the parallel zone. The spent zone of adsorbent may then be regenerated or the spent adsorbent may be replaced as desired.

Suitable adsorbent particles for the present invention are those adsorbent particles capable of selectively sorbing paraffin-insoluble compounds. Such adsorbents include hydrocarbon insoluble inorganic and carbonaceous materials, which have surface areas greater than 100 m²/g and whose surfaces may be acidic. Specific examples of adsorbents useful for this process include MCM-41, silica, silica-alumina, activated alumina, clays, e.g., fuller's earth, K-10 and similar acid-treated clays, and activated carbons. Preferably the surface areas are no greater than about 1000 m²/g. A suitable feedstream to adsorbent ratio is between 0.1 and 10 wt/wt. Such particles can range in size from about 5 mesh to about 100 mesh, say, from about 8 mesh to about 60 mesh.

The adsorption zone during regeneration can be maintained at a pressure from about 0 to about 2760 kPa (0 psig to about 400 psig), preferably from about 60 kPa to about 2070 kPa (10 psig to about 300 psig), a temperature from about 10° C. to about 400° C. (50° F. to about 752° F.), preferably from about 20° C. to about 300° C. (68° F. to about 572° F.) and a weight hourly space velocity from about 0.1 to about 1000, preferably from about 1 to about 500. The flow of the hydrocarbons through the adsorption zone may be conducted in an upflow, downflow or radial flow manner. The temperature and pressure of the adsorption zone are preferably selected to maintain the hydrocarbons in the liquid phase. After contacting the adsorbent for an appropriate time with the aromatics-containing desorber stream, the effluent is upgraded to a stream with lowered paraffin-insolubles content. The upgraded stream flows on to further refining processing, such as a thermal cracker, where it is treated either directly or blended with other refinery streams, such as conventional vacuum gas oil (VGO).

The swing reactor configuration can be set up such that one vessel is set up for adsorption, while the other vessel is set up for adsorbent regeneration. Regeneration of the adsorbent particles is carried out by using aromatics-containing desorber streams which may also include solvents such as toluene, toluene-methanol, or other appropriate solvents available in a refinery environment. The paraffin-insoluble molecules are washed off the adsorbent, and the resulting aromatics-containing stream containing these impurities is stripped in a suitable separation means such as a knockout drum and/or primary fractionator to provide an aromatics-rich stream and a paraffin insolubles-rich stream. Overhead from the knockout drum can be directed to the primary fractionator for further processing and recycling. The knockout drum bottoms are collected as tar which can be further treated by a coker, partial oxidation unit or other disposal technique.

When the adsorbent particles in an adsorber vessel become ineffective in removing paraffin-insoluble compounds from the feed, or otherwise deactivated, the vessel can be taken out of service and subjected to regeneration by passing an aromatics-containing stream over the adsorbent particles as noted above. This stream, which is at least partially derived from the cracked product stream of the present invention is directed to the adsorber vessel in regeneration mode under conditions sufficient to remove paraffin-insoluble compounds from the adsorbent particles. The adsorption zone during regeneration can be maintained under conditions the same or different from those employed during adsorption, e.g., at a pressure from about 0 to about 2760 kPa (0 psig to about 400 psig), preferably from about 60 kPa to about 2070 kPa (0 psig to about 400 psig, a temperature from about 10° to about 400° C. (50° F. to about 752° F.), preferably from about 20° C. to about 300° C. (68° F. to about 572° F.) and a weight hourly space velocity from about 0.1 to about 1000, preferably from about 1 to about 500. In addition to the aromatics-containing stream, the adsorbent particles can be treated with a suitable supplemental desorber stream, e.g., steam or carbon dioxide.

Aromatics-containing streams useful in the present invention to desorb adsorbent particles can typically contain from about 5 wt % to about 100 wt % aromatics as measured by aromatics-content determining techniques, e.g., gas chromatography, known to those skilled in the art. In certain embodiments of the present invention, the aromatics-containing stream can be supplied solely from aromatics-containing streams derived from crackate provided by the apparatus/process itself, e.g., steam-cracked gas oil, which typically contains from about 10 wt % to about 80 wt % aromatics. In alternate embodiments, the aromatics-containing stream is provided from outside the process, e.g., by providing externally supplied aromatics such as benzene, toluene and other commercially available aromatic compounds or mixtures thereof. Aromatics-containing product streams available from other parts of a refinery can also be used to supply the externally supplied aromatics-containing stream. In still other embodiments, the aromatics-containing stream can be provided by a mixture of aromatics-containing streams derived from the apparatus/process of the present invention itself, and an externally supplied source

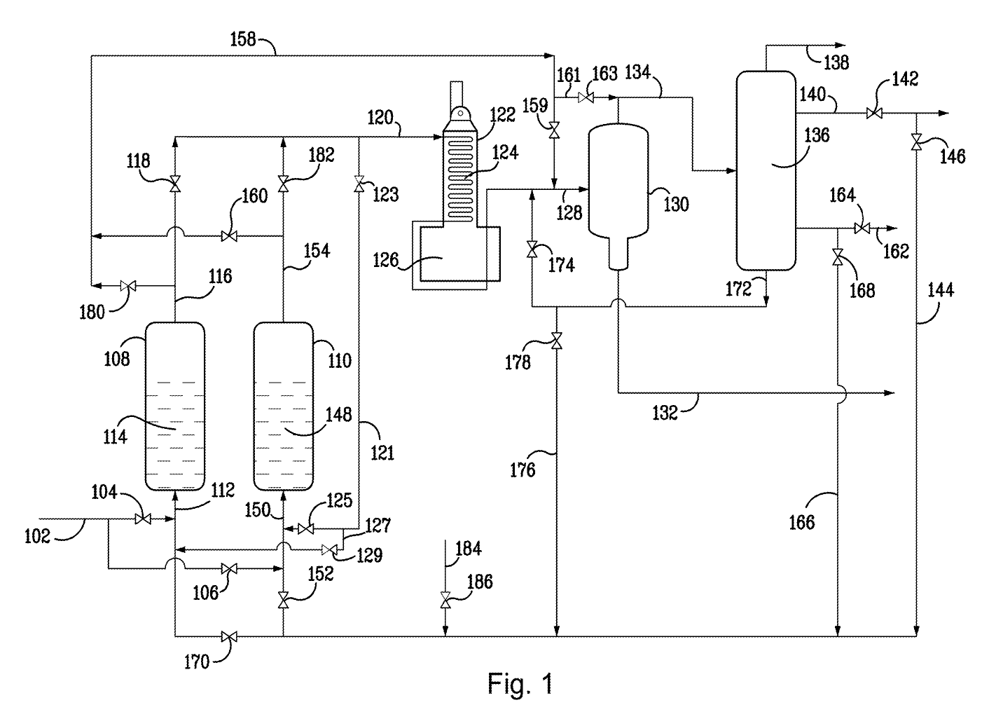

Reference is now made to accompanying FIG. 1 for a more detailed description and illustration of the invention.

In an embodiment of the present invention, a hydrocarbonaceous cracker feed stream obtained from condensate and containing about 70 ppm PI, is directed through line 102 controlled by feed inlet valves 104 and 106 which control the feed flow into adsorber vessels 108 or 110, which operate alternately in adsorption and regeneration modes, i.e., in swing configuration. When adsorber vessel 108 is in adsorption mode, hydrocarbonaceous feed passes though feed inlet 112 and contacts a fresh or regenerated adsorbent bed 114 packed with a suitable adsorbent such as activated carbon which removes at least some of the paraffin-insoluble compounds in the feed. Effluent diminished or depleted in paraffin-insoluble compounds content is directed through line 116 via valve 118 to the inlet 120 of a steam cracking furnace 122 comprising a convection zone 124 and a radiant zone 126.

The effluent from line 116 can be recycled to the adsorber vessel(s) via recycle line(s) 121 and/or 127 controlled by valves 123, 125, and 129 for further treatment to remove paraffin-insoluble compounds, as necessary.

Hot, cracked effluent passes from the cracking furnace via line 128 to tar knockout drum 130 and tar is taken as bottoms via line 132. The tar-depleted hot, cracked effluent is taken as overhead via line 134 to primary fractionator 136. The gaseous overhead of the primary fractionator is typically a C₄⁻ stream directed to a recovery train for recovering C₂ to C₄ olefins, inter alia. This stream is taken as overhead via line 138 with side streams of steam cracked naphtha (SCN) being taken as a side stream via line 140 controlled by valve 142. SCN can be cycled via line 144 controlled by valve 146 as an aromatics-containing desorber stream to adsorber vessel 110 which contains depleted adsorbent bed 148 via inlet 150 controlled by valve 152. The desorber stream contacts the adsorbent bed and removes aromatics-soluble paraffin-insoluble compounds which were earlier sorbed by the adsorbent bed during treatment of the hydrocarbonaceous feed from line 102 (which during adsorption mode operation is directed to the vessel via valve 106). The spent desorber stream passes out of the adsorption vessel 110 via line 154 to line 158 controlled by valve 160 and thence to the tar knockout drum 130 via line 128, for removal of the paraffin-insoluble compounds as tar. Alternately, the spent desorber stream can be directed to any separator apparatus which is capable of removing paraffin-insoluble compounds from the desorber stream as bottoms, e.g., a primary fractionator, membrane, or other separation means.

An additional aromatics-containing stream can be taken off the primary fractionator 136, as a steam cracked gas oil via line 162 controlled by valve 164. The steam cracked gas oil can also be cycled via line 166 controlled by valve 168 as an aromatics-containing desorber stream to line 144 and thence to adsorption vessel 108 or 110 (whichever is in the regeneration mode) via valve 170 or 152, respectively. Similarly, a bottoms fraction can be taken from the primary fractionator 136 via line 172 and directed to the tar knockout drum via valve 174 or as an aromatics-containing desorber stream to line 144 via line 176 controlled by valve 178. Thus, the aromatics-containing desorber stream can be derived from steam cracked naphtha, steam cracked gas oil, primary fractionator bottoms, or any combination thereof. A supplemental aromatics-containing desorber stream can be supplied to the adsorber vessel(s) from an external source (i.e., not derived from process crackates) via line 184 controlled by valve 186. In certain embodiments of the present invention, the external source can be used as the sole source of aromatics-containing desorber stream.

When adsorption vessel 108 swings into regeneration mode operation, the aromatics-containing desorber stream enters line 112 via valve 170. The desorber stream contacts the adsorbent bed and removes aromatics-soluble paraffin-insoluble compounds which were sorbed by the adsorbent bed during adsorption mode operation. The spent desorber stream passes out of the adsorption vessel 108 via line 116 to line 158 controlled by valves 159 and 180 and thence to the tar knockout drum 130 via line 128, for removal of the paraffin-insoluble compounds as tar. Alternatively, or supplementarily, the spent desorber stream can be directed to a suitable separator apparatus (other than the tar knockout drum) which is capable of removing paraffin-insoluble compounds from the desorber stream, e.g., directly to primary fractionator 130 via line 134 using line 161 controlled by valve 163. Suitable separators can include membranes as well as distillation columns.

When adsorption vessel 110 swings into adsorption mode operation, the hydrocarbonaceous feed enters line 150 via valve 106 and contacts a fresh or regenerated adsorbent bed 148 which removes at least some of the paraffin-insoluble compounds in the feed. Effluent diminished or depleted in paraffin-insoluble compounds is directed through line 154 via valve 182 to the inlet 120 of steam cracking furnace 122 and is treated in the manner discussed above.

The invention is further illustrated by the following Examples which are provided for the purpose of representation and are not to be construed as limiting the scope of the invention. Unless stated otherwise, all percentages, parts, etc., are by weight.

### EXAMPLE 1

### Removal of Paraffin-Insoluble Compounds from Hydrocarbonaceous Stream Using Activated Carbon

Separate hydrocarbon streams of naphtha, condensate and vacuum gas oil, respectively, were treated with activated carbon, i.e., APC carbon available from Calgon of Pittsburgh, Pa. USA. Hydrocarbon samples were mixed with carbon samples at a 4:1 weight ratio in a sealed vial. The naphtha/carbon and the condensate/carbon vials were kept at room temperature for 48 hours. The vacuum gas oil vial was kept at 75° C. (167° F.) for 24 hours. Results are provided in TABLE 1 below, along with the paraffin-insolubles concentration measured as pentane-insolubles before the carbon treatment. The results indicate that the APC activated carbon proves effective for removing paraffin-insoluble compounds from a range of hydrocarbonaceous feeds.

TABLE 1

PI, ppm

| SAMPLES | BEFORE | AFTER |
|---|---|---|
| Naphtha | 50-55 | 2-4 |
| Condensate | 23-23 | 1-2 |
| Vacuum Gas Oil | 127-137 | 53-39 |

### EXAMPLE 2

### Removal of Paraffin-Insoluble Compounds from Hydrocarbonaceous Stream Using Activated Carbons and Clays

A stream of condensate containing 67 ppm paraffin-insoluble compounds measured as pentane-insoluble were contacted with SG6 Carbon, CG6 Carbon (both carbons obtained from Cameron Carbon, Inc. of Havre de Grace, Md., USA), attapulgite clay, and Filtrol-24™ clay (an acid washed or activated montmorillonite clay obtainable from Engelhard Corporation). The treatments involved mixing a hydrocarbon sample and an adsorbent sample at a 4/1 weight ratio in a sealed vial. The vials were kept at room temperature 24 hours. Condensate was then recovered from the vials and analyzed for PI content. Fresh PI-containing condensate was then added to the same adsorbents at the same 4:1 ratio and the experiments were repeated. Twenty repetition tests were performed on CG6 Carbon and attapulgite clay; 14 repetition tests were performed on SG6 and Filtrol-24™ clay. After 20 treatments the CG6 Carbon-treated condensate showed only 2.5 ppm PI and the attapulgite clay-treated condensate showed 11.5 ppm PI, both of which were significantly lower than the 67 ppm value in the feed, indicating the effectiveness of the adsorbents' PI removal capability.

The present invention utilizes an adsorption process to remove a significant amount of paraffin-insoluble compounds from a hydrocarbonaceous feedstream, and to render that stream more valuable as a feed to a cracker. Any hydrocarbonaceous liquid containing paraffin-insoluble compounds, regardless of source, may be used for the feedstream of this invention. The present invention is especially useful inasmuch as it provides an aromatics-containing stream as a by-product of cracking, which stream is directed to an adsorption vessel operating in regeneration mode as a desorbent to remove paraffin-insoluble compounds from adsorbent particles. Such aromatics-containing streams can include gasoline, steam cracked naphtha, steam cracked gas oil, steam cracked quench oil, and primary fractionator bottoms which can be taken at various points downstream of a steam cracking furnace.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. An apparatus for cracking hydrocarbonaceous feed containing paraffin-insoluble compounds, which comprises:
A) a feed treating zone, which comprises an adsorber vessel for treating the hydrocarbonaceous feed comprising a) a hydrocarbonaceous feed inlet, b) adsorbent particles capable of selectively sorbing paraffin-insoluble compounds, and c) an outlet for removing paraffin-insoluble compounds-depleted effluent;
B) a cracking zone comprising a) an inlet for receiving paraffin-insoluble compounds-depleted effluent from the feed treating zone, b) an outlet for removing cracked effluent containing aromatics;
C) a separator for removing an aromatics-containing stream from the cracked effluent comprising a) an inlet for receiving the cracked effluent, b) an outlet for removing the aromatics-containing stream, and c) an outlet for removing remaining cracked effluent; and
D) an inlet to the feed treating zone for receiving aromatics-containing stream as desorbent from the outlet for removing the aromatics-containing stream of step C).

2. The apparatus of claim 1 wherein the separator C) comprises a primary fractionator.

3. The apparatus of claim 2 which further comprises E) a tar knockout drum between B) and C), comprising a) an inlet for receiving cracked effluent containing aromatics, b) a bottom outlet for removing tar and c) an upper outlet for directing tar-depleted effluent to the primary fractionator.

4. The apparatus of claim 3 wherein the primary fractionator comprises a) an inlet for receiving the tar-depleted effluent, b) a bottoms outlet for removing fractionator bottoms, and c) at least one outlet for removing lower boiling products.

5. The apparatus of claim 4 wherein the primary fractionator comprises d) an overhead outlet, e) an upper outlet for removing steam-cracked naphtha and f) a lower outlet for removing steam-cracked gas oil.

6. The apparatus of claim 5 wherein at least one of the upper outlet for removing steam-cracked naphtha and lower outlet for removing steam-cracked gas oil communicates with D) to provide an aromatics-containing desorber stream.

7. The apparatus of claim 1 wherein the feed treating zone comprises at least two adsorption vessels arranged to allow selectively sorbing paraffin-insoluble compounds by adsorbent particles in one vessel while regenerating adsorbent particles in another vessel to provide an aromatics-containing stream containing desorbed paraffin-insoluble compounds, each vessel having an outlet for removing the aromatics-containing stream containing desorbed paraffin-insoluble compounds.

8. The apparatus of claim 7 wherein an outlet in the adsorption vessels for removing the aromatics-containing stream containing desorbed paraffin-insoluble compounds communicates with the tar knockout drum inlet.

9. The apparatus of claim 4 wherein the bottoms outlet for removing primary fractionator bottoms communicates with the tar knockout drum inlet.

10. The apparatus of claim 1 wherein the adsorbent particles contain components selected from the group consisting of activated carbon, MCM-41, activated alumina, silica, aluminosilicate, clay, and fuller's earth.

11. The apparatus of claim 1 which further comprises a line for recycling the paraffin-insoluble compounds-depleted effluent to the hydrocarbonaceous feed inlet of the adsorber vessel.

12. A process for cracking hydrocarbonaceous feed containing paraffin-insoluble compounds that comprises:
a) contacting the feed in an adsorption zone with adsorbent particles capable of selectively adsorbing paraffin insoluble compounds from the feed under conditions sufficient for the adsorbent particles to sorb the paraffin-insoluble compounds to provide a treated feed of reduced paraffin-insoluble compounds content and adsorbent particles at least partially sorbed with paraffin-insoluble compounds;
b) cracking the treated feed in a cracking zone at conditions sufficient to provide a cracked product containing aromatics;
c) separating an aromatics-containing stream from the cracked product; and
d) directing the aromatics-containing stream from the cracked product to the adsorption zone and contacting with the adsorbent particles under desorption conditions sufficient to at least partially desorb the paraffin-insoluble compounds therefrom, thereby providing regenerated adsorbent particles and an aromatics-containing stream containing desorbed paraffin-insoluble compounds.

13. The process of claim 12 which further comprises:
e) separating the regenerated adsorbent particles from the aromatics-containing stream containing desorbed paraffin-insoluble compounds.

14. The process of claim 13 which further comprises:
f) separating the paraffin insoluble compounds from the aromatics-containing stream containing desorbed paraffin insoluble compounds.

15. The process of claim 14 which further comprises:
g) recycling the aromatics-containing stream of step f) to step d).

16. The process of claim 12 wherein the hydrocarbonaceous feed containing paraffin-insoluble compounds is selected from the group consisting of condensate and gas oil, the adsorbent particles contain components selected from the group consisting of activated carbon, MCM-41, activated alumina, silica, aluminosilicate, clay, and fuller's earth, and the paraffin-insoluble compounds are selected from the group consisting of pentane-insoluble compounds and heptane-insoluble compounds.

17. The process of claim 16 wherein the adsorption zone comprises at least two adsorption vessels which provide for simultaneous operation of steps a) and d) in separate adsorption vessels.

18. The process of claim 12 wherein the aromatics-containing stream from the lower boiling cracked product is selected from the group consisting of gasoline, toluene, xylene, steam-cracked naphtha, steam-cracked gas oil, and steam-cracked quench oil.

19. The process of claim 14 wherein the separation in step f) is carried out in at least one of a tar knockout drum and a primary fractionator.

20. The process of claim 12 which further comprises:
h) recycling at least a portion of the treated feed to the adsorption zone.

21. An apparatus for steam cracking hydrocarbonaceous feed containing paraffin-insoluble compounds, which comprises:
A) a feed treating zone, which comprises an adsorber vessel for treating the hydrocarbonaceous feed comprising a) a hydrocarbonaceous feed inlet, b) adsorbent particles capable of selectively sorbing paraffin-insoluble compounds, and c) an outlet for removing paraffin-insoluble compounds-depleted effluent;
B) a steam cracking zone comprising a) an inlet for receiving paraffin-insoluble compounds-depleted effluent from the feed treating zone, b) an outlet for removing cracked effluent containing aromatics, and c) a steam inlet; and
C) a separator for removing an aromatics-containing stream from the cracked effluent comprising a) an inlet for receiving the cracked effluent, b) an outlet for removing the aromatics-containing stream, and c) an outlet for removing remaining cracked effluent.

22. The apparatus of claim 21 wherein the adsorber vessel further comprises: at least one of d) an inlet to the feed treating zone for receiving aromatics-containing stream as desorbent, and e) a line for recycling paraffin-insolubles compounds-depleted effluent from outlet c) to hydrocarbonaceous feed inlet a).

23. A process for steam cracking hydrocarbonaceous feed containing paraffin-insoluble compounds that comprises:
a) contacting the feed in an adsorption zone with adsorbent particles capable of selectively adsorbing paraffin insoluble compounds from the feed under conditions sufficient for the adsorbent particles to sorb the paraffin-insoluble compounds to provide a treated feed of reduced paraffin-insoluble compounds content and adsorbent particles at least partially sorbed with paraffin-insoluble compounds;
b) steam cracking the treated feed in a cracking zone at conditions sufficient to provide a cracked product containing aromatics; and
c) separating an aromatics-containing stream from the cracked product.

24. The process of claim 23 which further comprises:
d) directing an aromatics-containing stream to the adsorption zone and contacting with the adsorbent particles under desorption conditions sufficient to at least partially desorb the paraffin-insoluble compounds therefrom, thereby providing regenerated adsorbent particles and an aromatics-containing stream containing desorbed paraffin-insoluble compounds.

25. The process of claim 23 which further comprises e) recycling at least a portion of the treated feed of reduced paraffin-insoluble compounds content to the adsorption zone.
